(19) United States
(12) Patent Application Publication
(10) Pub. No.: US 2025/0366795 A1
(43) Pub. Date: Dec. 4, 2025

(54) PHYSIOLOGICAL PARAMETER DISPLAY

(71) Applicant: Hill-Rom Services, Inc., Batesville, IN (US)

(72) Inventors: Branden Dundey, Jupiter, FL (US); Matthew Vincent, Jupiter, FL (US)

(21) Appl. No.: 18/731,978

(22) Filed: Jun. 3, 2024

(51) Int. Cl. A61B 5/00; A61B 5/0205; A61B 5/0215; A61B 5/024; A61B 5/026; A61B 5/08; A61B 5/1455; A61B 5/339; G16H 40/63

(52) U.S. Cl. CPC A61B 5/743; A61B 5/002; A61B 5/02055; A61B 5/339; A61B 5/7435; G16H 40/63; A61B 5/02152; A61B 5/024; A61B 5/026; A61B 5/0816; A61B 5/14551

(57) ABSTRACT

A device for displaying physiological parameter measurements is disclosed. The device displays a graphical user interface including playback of a plurality of waveforms. Each waveform of the plurality of waveforms representing a physiological parameter measured over a period of time. The plurality of waveforms including at least: a first waveform representing a first physiological parameter; and a second waveform representing a second physiological parameter. The device receives a settings adjustment on the graphical user interface. The device adjusts the playback of the second waveform relative to the first waveform based on the settings adjustment. The playback of the second waveform is adjusted to change a sweep speed of the second waveform such that a length of the second waveform differs from a length of the first waveform over the period of time.

### BACKGROUND

Medical devices collect, monitor, and display various aspects associated with a patient's physiology. Physiological data acquired from the medical devices can be maintained in a database for a timeline determined by a medical facility to support clinical case review, research, alarm analytics, and the quality control objectives of the medical facility. In addition, the physiological data can be exported and/or imported in an HL7/XML format to and from electronic medical record (EMR) systems or other systems specified by the medical facility.

### SUMMARY

In general terms, the present disclosure relates to displaying physiological parameter measurements. In one possible configuration, a playback speed of a waveform representing a physiological parameter is adjusted relative to the playback speeds of different waveforms representing different physiological parameters. Various aspects are described in this disclosure, which include, but are not limited to, the following aspects.

One aspect relates to a device for displaying physiological parameter measurements, the device comprising: at least one processing device; and at least one computer-readable data storage device storing software instructions that, when executed by the at least one processing device, cause the at least one processing device to: display a graphical user interface including playback of a plurality of waveforms, each waveform of the plurality of waveforms representing a physiological parameter measured over a period of time, the plurality of waveforms including at least: a first waveform representing a first physiological parameter; and a second waveform representing a second physiological parameter; receive a settings adjustment on the graphical user interface; and adjust the playback of the second waveform relative to the first waveform based on the settings adjustment, wherein the playback of the second waveform is adjusted to change a sweep speed of the second waveform such that a length of the second waveform differs from a length of the first waveform over the period of time.

Another aspect relates to a method of displaying physiological parameter measurements, the method comprising: displaying a graphical user interface including playback of a plurality of waveforms, each waveform of the plurality of waveforms representing a physiological parameter measured over a period of time, the plurality of waveforms including at least: a first waveform representing a first physiological parameter; and a second waveform representing a second physiological parameter; receiving a settings adjustment on the graphical user interface; and adjusting the playback of the second waveform relative to the first waveform based on the settings adjustment, the playback of the second waveform being adjusted to change a sweep speed of the second waveform such that a length of the second waveform differs from a length of the first waveform over the period of time.

Another aspect relates to non-transitory computer-readable media storing data instructions, which when executed by one or more processing devices, cause the one or more processing devices to: display a graphical user interface including playback of a plurality of waveforms, each waveform of the plurality of waveforms representing a physiological parameter measured over a period of time, the plurality of waveforms including at least: a first waveform representing a first physiological parameter; and a second waveform representing a second physiological parameter; receive a settings adjustment on the graphical user interface; and adjust the playback of the second waveform relative to the first waveform, wherein the playback of the second waveform is adjusted to change a sweep speed of the second waveform such that a length of the second waveform differs from a length of the first waveform over the period of time.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for visualizing physiological data captured from a patient in a patient environment.

FIG. 2 schematically illustrates examples of a patient support apparatus, a patient monitoring device, and a data visualization system of the system of FIG. 1

FIG. 3 schematically illustrates an example of a method of displaying physiological parameter measurements that can be performed on a workstation monitor by the data visualization system of FIG. 2.

FIG. 4 illustrates an example of a graphical user interface that can be displayed on the workstation monitor by the data visualization system of FIG. 2.

FIG. 5 illustrates an example of a graphical user interface that can be displayed on the workstation monitor by the data visualization system of FIG. 2.

FIG. 6 illustrates an example of a graphical user interface that can be displayed on the workstation monitor by the data visualization system of FIG. 2.

FIG. 7 illustrates an example of a graphical user interface that can be displayed on the workstation monitor by the data visualization system of FIG. 2.

FIG. 8 illustrates another example of a graphical user interface that can be displayed on the workstation monitor by the data visualization system of FIG. 2.

FIG. 9 illustrates another example of a graphical user interface that can be displayed on the workstation monitor by the data visualization system of FIG. 2.

FIG. 10 illustrates another example of a graphical user interface that can be displayed on the workstation monitor by the data visualization system of FIG. 2.

FIG. 11 illustrates another example of a graphical user interface that can be displayed on the workstation monitor by the data visualization system of FIG. 2.

FIG. 12 illustrates another example of a graphical user interface that can be displayed on the workstation monitor by the data visualization system of FIG. 2.

FIG. 13 illustrates another example of a graphical user interface that can be displayed on the workstation monitor by the data visualization system of FIG. 2.

### DETAILED DESCRIPTION

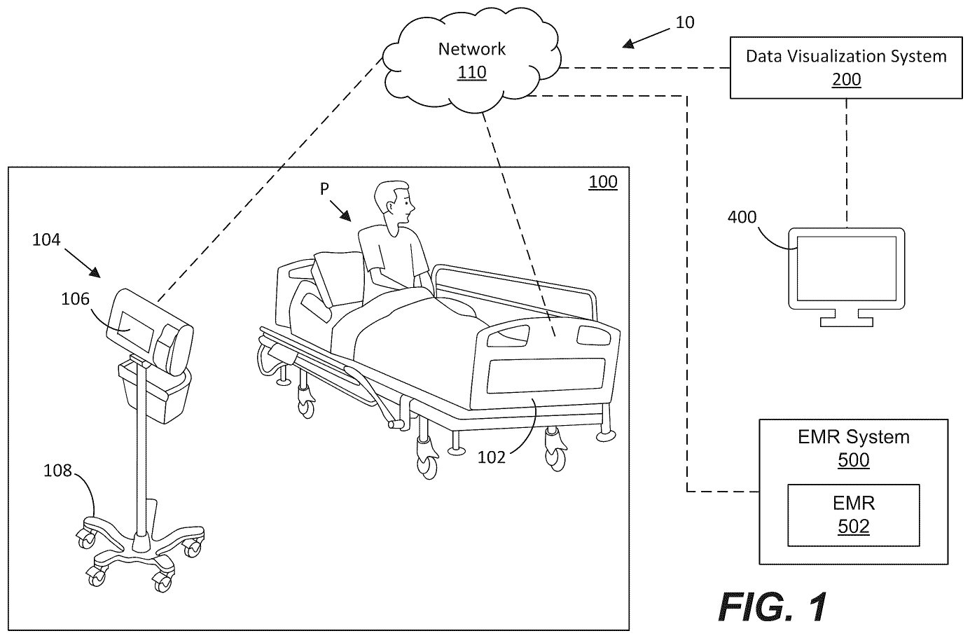

FIG. 1 illustrates an example of a system 10 for visualizing physiological data captured from a patient P in a patient environment 100. The patient P is shown resting on a patient support apparatus 102 inside the patient environment 100. The patient environment 100 can be an area within a medical facility such as a patient room in a hospital. The patient environment 100 includes medical equipment such as the patient support apparatus 102, and a patient monitoring device 104 that can be used to capture the physiological data.

As shown in FIG. 1, the patient P is supported on the patient support apparatus 102 inside the patient environment 100. The patient support apparatus 102 can be a hospital bed, a stretcher, operating room table, or similar type of apparatus on which the patient P can rest. The patient support apparatus 102 can include one or more sensors that measure one or more physiological parameters of the patient P such as respiration rate, heart rate, non-invasive blood pressure (NIBP), motion, and weight. Additionally, the patient support apparatus 102 can include sensors that detect patient exit, incontinence, deterioration, and other metrics.

The patient monitoring device 104 can be used to measure and monitor physiological parameters of the patient P. The patient monitoring device 104 displays representations of the measured physiological parameters including numerical values and waveforms on a display 106. In some examples, the display 106 includes a touchscreen that operates to receive tactile inputs from a user such as a caregiver such that the display 106 is both a display device and a user input device. In some examples, the display 106 is a liquid-crystal display (LCD), an organic light-emitting diode (OLED, a plasma panel, a quantum-dot light-emitting diode (QLED), or other type or combination of display screen technology.

In the illustrative example shown in FIG. 1, the patient monitoring device 104 is mounted on a mobile cart 108 such that the patient monitoring device 104 is portable and can be brought into and out of the patient environment 100. In alternative examples, the patient monitoring device 104 can be stationary such that it can include a wall mounted unit.

As shown in FIG. 1, the patient support apparatus 102 and the patient monitoring device 104 are connected to a network 110. The network 110 can connect and exchange data between the patient support apparatus 102 and the patient monitoring device 104 and other equipment inside the patient environment 100. Further, the network 110 can connect and exchange data between the patient support apparatus 102 and the patient monitoring device 104 and other systems and devices outside of the patient environment 100. The network 110 can include any type of wired or wireless connections, or any combinations thereof. The wireless connections can be accomplished using Wi-Fi, ultra-wideband (UWB), Bluetooth, and the like. In some examples, the network 110 is an Internet of things (IoT) network.

As further shown in FIG. 1, the network 110 transfers the physiological parameter data captured by the patient support apparatus 102, the patient monitoring device 104, and other medical devices in the patient environment 100 to a data visualization system 200 for display on a workstation monitor 400. The workstation monitor 400 is an example of a device for displaying physiological parameter measurements captured by the patient support apparatus 102, the patient monitoring device 104, and other medical devices inside the patient environment 100.

In some examples, the data visualization system 200 is communicatively connected to the workstation monitor 400 via the network 110. Alternatively, the data visualization system 200 can be connected directly to the workstation monitor 400 via wired and/or wireless connections without using the network 110 to communicate with the workstation monitor 400.

The data visualization system 200 can be used to transfer, store, and/or convert the physiological parameter data of the patient P captured by the patient support apparatus 102, the patient monitoring device 104, and other medical devices inside the patient environment 100. Further, the data visualization system 200 displays the physiological parameter data captured by the patient support apparatus 102, the patient monitoring device 104, and other medical devices on the workstation monitor 400. The data visualization system 200 can be used for post-acquisition data review, quality improvement, and research purposes.

The data visualization system 200 can acquire the physiological parameter data from the patient support apparatus 102, the patient monitoring device 104, and other medical devices for long-term storage and distribution to external systems such as an Electronic Medical Records (EMR) system 500 that maintains an EMR 502 of the patient P.

As described herein, the terms electronic health records (EHRs) and electronic patient record (EPRs) can be used interchangeably with EMRs. The EMR system 500 collects electronic health information of the patient P in a digital format for storage in the EMR 502. The EMR system 500 maintains a plurality of EMRs 502 for a plurality of patients. Each EMR 502 can be shared across different health care settings. For example, the EMRs 502 can be shared through network-connected, enterprise-wide information systems or other information networks and exchanges. The EMRs 502 may include a range of data, including demographics, medical history, medication and allergies, immunization status, laboratory test results, radiology images, vital signs, personal statistics like age and weight, and billing information.

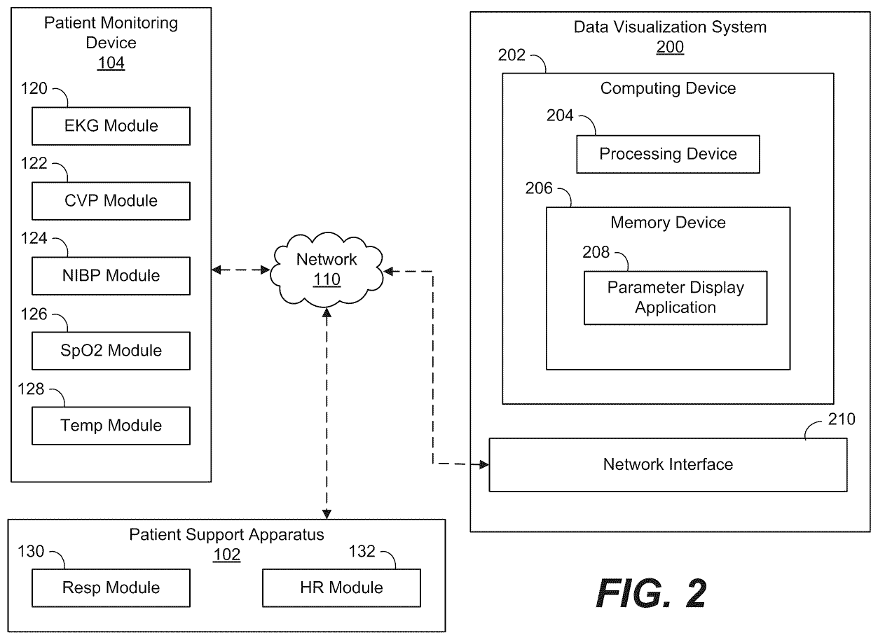

FIG. 2 schematically illustrates examples of the patient support apparatus 102, the patient monitoring device 104, and the data visualization system 200. The patient monitoring device 104 includes one or more sensor modules that can be used to measure one or more physiological parameters of the patient P. As used herein, a “module” is a combination of physical structure which resides in the patient monitoring device 104 and peripheral components that attach to and reside outside of the patient monitoring device 104.

As shown in the examples of FIG. 2, the patient monitoring device 104 can include an EKG sensor module 120 that can be used to measure and record electrocardiogram signals of the patient P's heart activity. The patient monitoring device 104 can include a central venous pressure (CVP) sensor module 122 that can be used to measure and record CVP data of the patient P, which is the pressure in the vena cavae, near the right atrium of the heart. The patient monitoring device 104 can include a non-invasive blood pressure (NIBP) sensor module 124 that can be used to measure and record the patient P's blood pressure. The patient monitoring device 104 can include a pulse oximetry sensor module 126 that can be used to measure and record the patient P's blood oxygen saturation (SpO2) and pulse. The patient monitoring device 104 can include a temperature sensor module 128 that can be used to measure and record the patient P's temperature. The patient monitoring device 104 can include additional types of sensor modules for measuring additional types of physiological parameters of the patient P, as desired.

As further shown in the examples of FIG. 2, the patient support apparatus 102 can include one or more sensor modules for measuring and recording additional types of physiological parameters of the patient P. For example, the patient support apparatus 102 can include a respiration sensor module 130 that can measure and record the respiration rate of the patient P. The patient support apparatus 102 can further include a heart rate sensor module 132 that can measure and record the heart rate of the patient P. In some examples, the respiration sensor module 130 and the heart rate sensor module 132 are implemented in a contact-free, continuous monitoring pad that is placed under a mattress of the patient support apparatus 102. Alternatively, the respiration sensor module 130 and the heart rate sensor module 132 can be integrated into a frame of the patient support apparatus 102.

As further shown in FIG. 2, the data visualization system 200 includes a computing device 202 having at least one processing device 204 and a memory device 206. The at least one processing device 204 is an example of a processing unit such as a central processing unit (CPU). The at least one processing device 204 can include one or more central processing units (CPUs). In some examples, the at least one processing device 204 includes one or more digital signal processors, field-programmable gate arrays, and/or other types of electronic circuits.

The memory device 206 operates to store data and instructions for execution by the at least one processing device 204. In the example illustrated in FIG. 2, the memory device 206 stores a waveform display application 208, which will be described in more detail. The memory device 206 includes computer-readable media, which may include any media that can be accessed by the at least one processing device 204. By way of example, computer-readable media include computer-readable storage media and computer-readable communication media. As such, the memory device 206 is an example of a computer-readable data storage device storing software instructions for execution by the at least one processing device 204.

Computer-readable storage media includes volatile and nonvolatile, removable and non-removable media implemented in any device configured to store information such as computer-readable instructions, data structures, program modules, or other data. Computer-readable storage media can include, but is not limited to, random access memory, read only memory, electrically erasable programmable read only memory, flash memory, and other memory technology, including any medium that can be used to store information that can be accessed by the camera. The computer-readable storage media is non-transitory.

Computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

The data visualization system 200 further includes a network interface 210 that allows the data visualization system 200 to connect to the network 110. The network interface 210 can include wired interfaces and/or wireless interfaces. For example, the network interface 210 can wirelessly connect to the network 110 through Wi-Fi, or other wireless connections. Alternatively, the network interface 210 can connect to the network 110 using wired connections such as through an Ethernet or Universal Serial Bus (USB) cable.

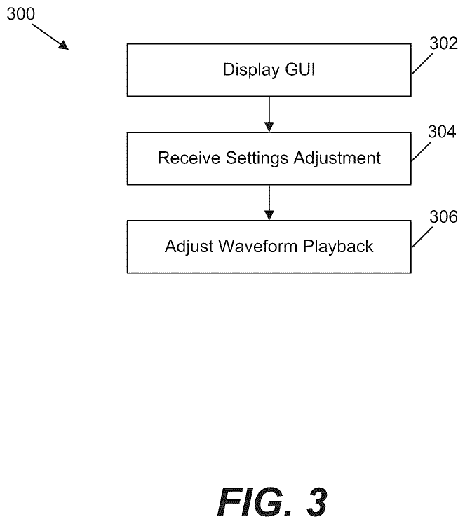

FIG. 3 schematically illustrates an example of a method 300 of displaying physiological parameter measurements that can be performed on the workstation monitor 400 by the data visualization system 200. The method 300 can be performed by the at least one processing device 204 executing the waveform display application 208 which is stored on the memory device 206 of the data visualization system 200.

As shown in FIG. 3, the method 300 includes an operation 302 of displaying a graphical user interface that includes playback of a plurality of waveforms, each waveform of the plurality of waveforms representing a physiological parameter measured over a period of time. The playback of the plurality of waveforms can resemble a digital video recorder (DVR) playback where an end user can rewind and playback the waveforms as desired for post-acquisition data review, quality improvement, and research purposes.

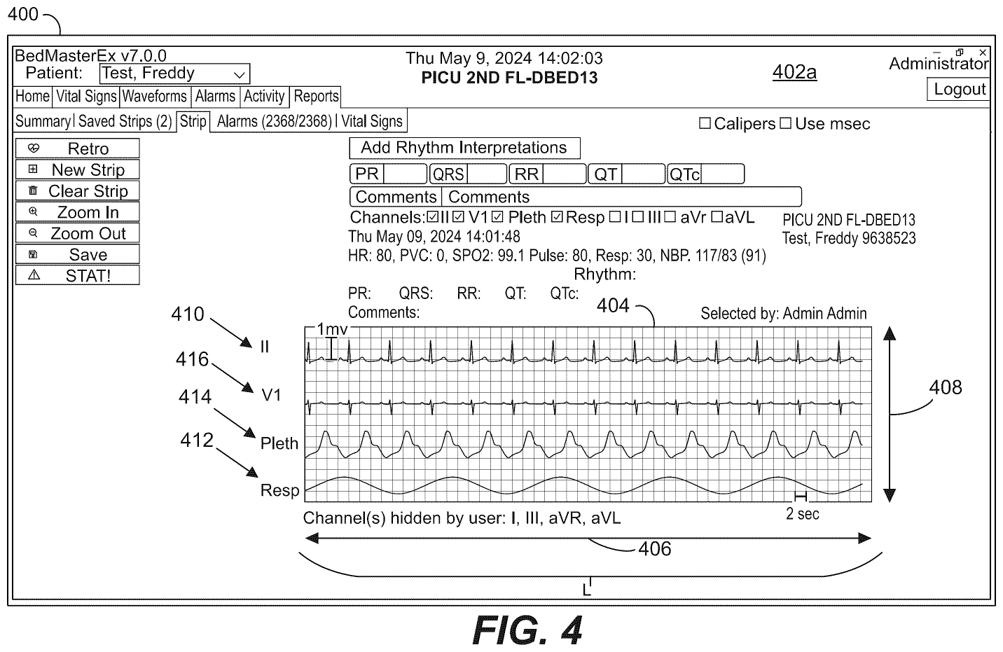

FIG. 4 illustrates an example of a graphical user interface 402a that can be displayed on the workstation monitor 400 by the data visualization system 200 in accordance with operation 302 of the method 300. The graphical user interface 402a is an example of a report page that can be generated by the data visualization system 200. The report page provides a dynamic tool for documenting cardiac monitoring in a fully digital format such that the report page eliminates the need for paper strips, and can be used to build reports that include multi-channel waveforms such as a plurality of waveforms displayed in a waveform display area 404 of the graphical user interface 402a. The waveforms displayed in the waveform display area 404 are charted over a period of time on an X-axis 406 and have an amplitude on a Y-axis 408. In some examples, the X-axis represents a period of time of about 10 seconds.

In the example shown in FIG. 4, the waveform display area 404 includes at least a first waveform 410 representing a first physiological parameter, and a second waveform 412 representing a second physiological parameter. In the example shown in FIG. 4, the first waveform 410 represents an electrocardiogram signal captured by a lead II of the EKG sensor module 120 of the patient monitoring device 104, and the second waveform represents a respiration rate that can be captured by a respiration rate sensor within the patient environment 100 such as the respiration sensor module 130 of the patient support apparatus 102.

As used herein, sweep speed refers to the time required to complete one sweep of data, such as the speed at which the EKG sensor module 120 records the electrocardiogram data over time. The sweep speed of the respiration rate data from the respiration sensor module 130 is slower than the sweep speed of the electrocardiogram data the EKG sensor module 120. For example, the sweep speed of the respiration sensor module 130 is 6.25 mm/second and the sweep speed of the EKG sensor module 120 is 25 mm/second.

As further used herein, playback speed refers to the speed of a waveform played back across the X-axis 406 of the waveform display area 404. The playback speed is also expressed in a unit of length per second such as millimeters per second (mm/sec). In instances where the playback speed matches the sweep speed of a sensor module, the waveforms when played back in the waveform display area 404 appear as they would on the display 106 of the patient monitoring device 104. However, when the playback speed differs from the sweep speed of a sensor module, the waveforms when played back in the waveform display area 404 will have a different appearance as they would on the display 106 of the patient monitoring device 104.

In some instances, it is desirable for the first and second waveforms 410, 412 to have the same length along the X-axis 406 to facilitate comparisons between the first and second waveforms 410, 412. In the example shown in FIG. 4, the second waveform 412 has the same length L as the first waveform 410 along the X-axis 406. This can be accomplished by adjusting the playback of the second waveform 412 to have the same playback speed as the first waveform 410. For example, the playback speed of the respiration rate can be increased to 25 mm/second even though the sweep speed of the respiration sensor module 130 is 6.25 mm/second such that the length of the second waveform 412 matches the length of the first waveform 410 during playback of the first and second waveforms 410, 412 in the waveform display area 404.

However, increasing the playback speed of the second waveform 412 relative to the sweep speed of the respiration sensor module 130 causes the second waveform 412 to have a stretched appearance which visually differs from the respiration rate waveforms typically displayed on the display 106 of the patient monitoring device 104. In some instances, the stretched appearance is undesirable because it looks unfamiliar and is more difficult to interpret.

As further shown in FIG. 4, the waveform display area 404 can further include a third waveform 414 representing a third physiological parameter such as a plethysmograph or “pleth” which is a measure of volumetric changes associated with pulsatile arterial blood flow measured by the pulse oximetry sensor module 126. The waveform display area 404 can also further include a fourth waveform 416 representing a fourth physiological parameter such as an electrocardiogram signal captured by a lead V1 of the EKG sensor module 120 of the patient monitoring device 104. The pulse oximetry sensor module 126 can have the same sweep speed of the EKG sensor module 120 (e.g., about 25 mm/second) such that the third waveform 414 has the same length as the first and fourth waveforms 410, 416 without having a stretched appearance unlike the second waveform 412 captured by the respiration sensor module 130.

Referring back to FIG. 3, the method 300 includes an operation 304 of receiving a settings adjustment on the graphical user interface 402 displayed on the workstation monitor 400.

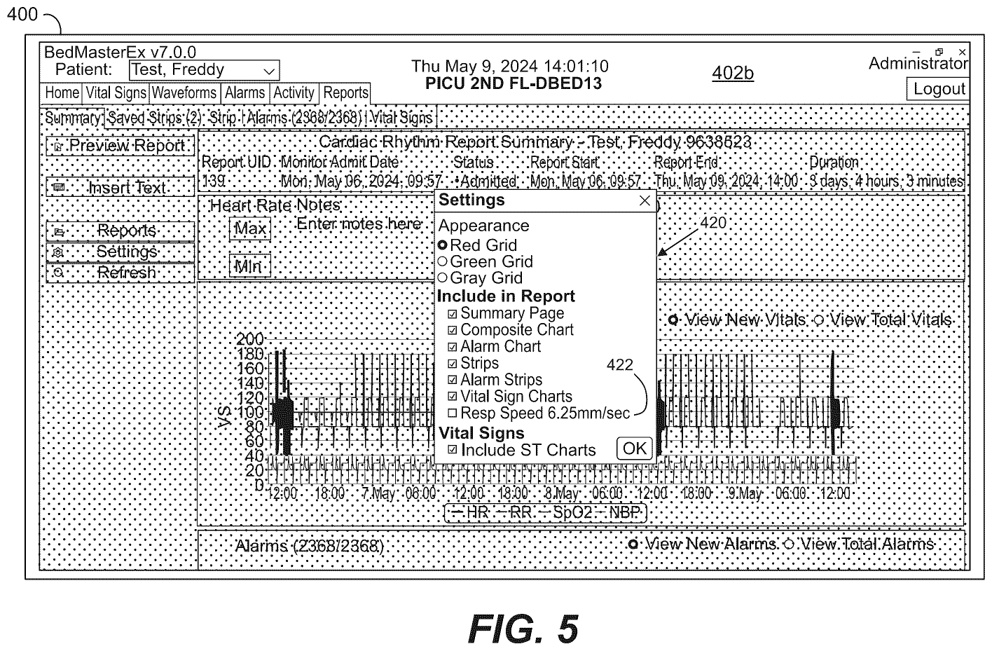

FIG. 5 illustrates an example of a graphical user interface 402b that can be displayed on the workstation monitor 400 by the data visualization system 200 in accordance with the method 300. As shown in FIG. 5, a settings menu 420 is displayed on the graphical user interface 402b. The settings menu 420 includes one or more options for selection by a user related to the appearance of a report generated from the report page, what to include in the report generated from the report page, and what types of vital signs to include in the report generated from the report page. In FIG. 5, a settings adjustment 422 for respiration rate playback speed (e.g., 6.25 mm/second) is unchecked such that the second waveform 412 has a playback speed (e.g., 25 mm/second) that matches the playback speed of the other waveforms displayed in the waveform display area 404. As discussed above, by having the playback speed of the second waveform 412 match the playback speed of the first waveform 410, the second waveform 412 has the same length L as the first waveform 410 which can be desirable in some instances. However, by making the second waveform 412 to have the same length L as the first waveform 410, the second waveform 412 has a stretched appearance which can be undesirable in other instances.

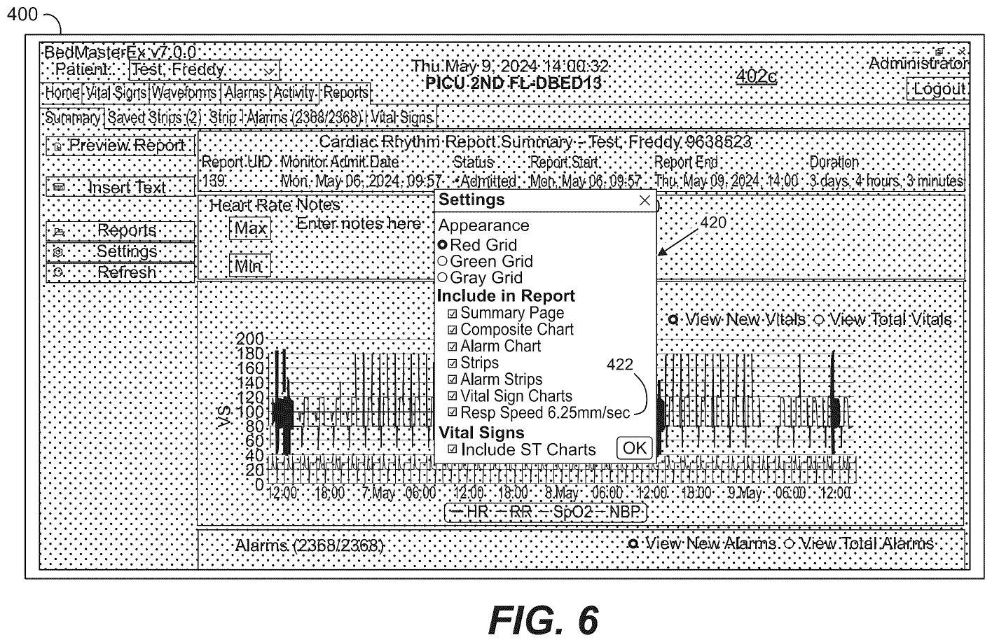

FIG. 6 illustrates an example of a graphical user interface 402c that can be displayed on the workstation monitor 400 by the data visualization system 200 in accordance with the method 300. In this example, the settings adjustment 422 for respiration rate playback speed (e.g., 6.25 mm/second) is checked such that the second waveform 412 has a slower playback speed than the other waveforms displayed in the waveform display area 404. Accordingly, the operation 304 of receiving the settings adjustment on the graphical user interface 402 displayed on the workstation monitor 400 can include receiving a checking or an unchecking of the settings adjustment 422 in the settings menu 420 for adjusting the playback speed of the second waveform 412 relative to the other waveforms in the waveform display area 404.

Referring back to FIG. 3, the method 300 includes an operation 306 of adjusting the playback of the second waveform 412 relative to the first waveform 410 based on the settings adjustment received in operation 304. In operation 306, the playback of the second waveform 412 can be adjusted to decrease the playback speed of the second waveform 412 such as when the settings adjustment 422 is checked (see FIG. 6). This causes the length of the second waveform 412 to differ from the length of the first waveform 410 over the period of time displayed along the X-axis 406 in the waveform display area 404 of the graphical user interface 402. The decreased length of the second waveform 412 more accurately mimics the respiration rate waveforms displayed on the display 106 of the patient monitoring device 104. In instances where the second waveform 412 is representative of respiration rate, the playback speed of the second waveform 412 is decreased from 25 mm/second to 6.25 mm/second.

Alternatively, operation 306 can include adjusting the playback of the second waveform 412 to increase the playback speed of the second waveform 412 such that the length of the second waveform 412 matches the length of the first waveform 410 over the period of time displayed in the waveform display area 404. This occurs when the settings adjustment 422 in the settings menu 420 is adjusted from checked to unchecked (see FIG. 5). In instances where the second waveform 412 is representative of respiration rate, the sweep speed of the second waveform 412 can increased from 6.25 mm/second to 25 mm/second such that the second waveform 412 has a playback speed that matches the playback speed of waveforms captured by the EKG sensor module 120 and other sensor modules.

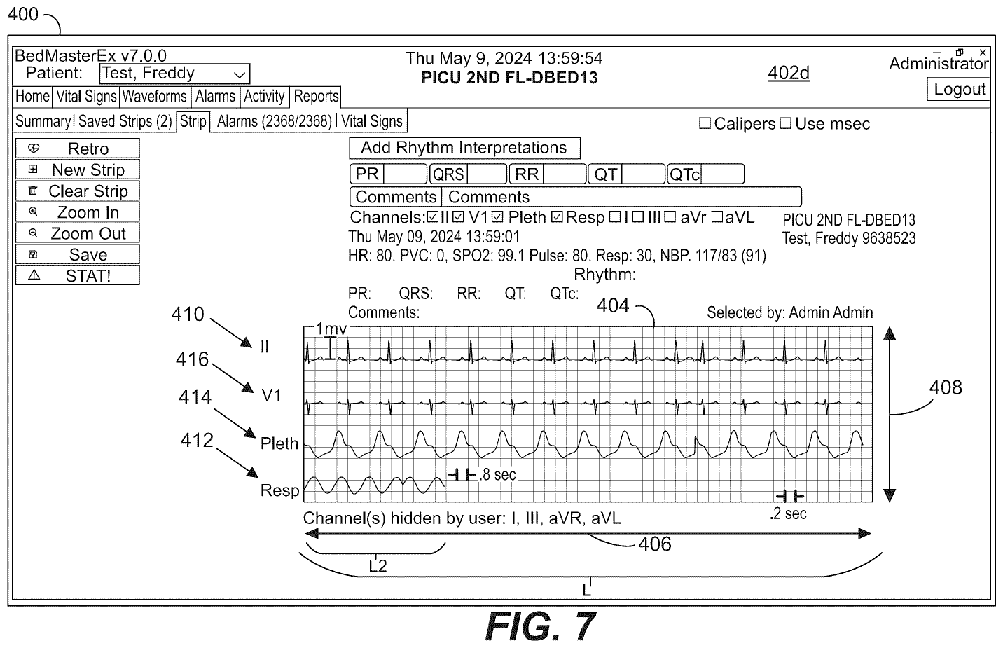

FIG. 7 illustrates an example of a graphical user interface 402d that can be displayed on the workstation monitor 400 by the data visualization system 200 in accordance with operation 306 of the method 300. FIG. 7 is another example of a report page that can be generated by the data visualization system 200. In this example, the length L2 of the second waveform 412 is shorter than the length L1 of the first waveform 410 due to the slower playback speed of the second waveform 412 relative to the first waveform 410. As shown in FIG. 7, the appearance of the second waveform 412 more closely mimics the respiration rate waveform displayed on the display 106 of the patient monitoring device 104.

As shown in the example provided in FIG. 7, the playback speed of the second waveform 412 is also adjusted relative to the third waveform 414 based on the settings adjustment received on the graphical user interface 402c of FIG. 6 such that the length L2 of the second waveform differs (e.g., is shorter) from a length of the third waveform 414 over the period of time along the X-axis 406. Also, the playback speed of the second waveform 412 is also adjusted relative to the fourth waveform 416 based on the settings adjustment received on the graphical user interface 402c of FIG. 6 such that the length L2 of the second waveform 412 also differs (e.g., is shorter) from a length of the fourth waveform 416 over the period of time along the X-axis 406. Accordingly, the playback of the second waveform 412 can be adjusted independently of the playback of the first, third, and fourth waveforms 410, 414, 416.

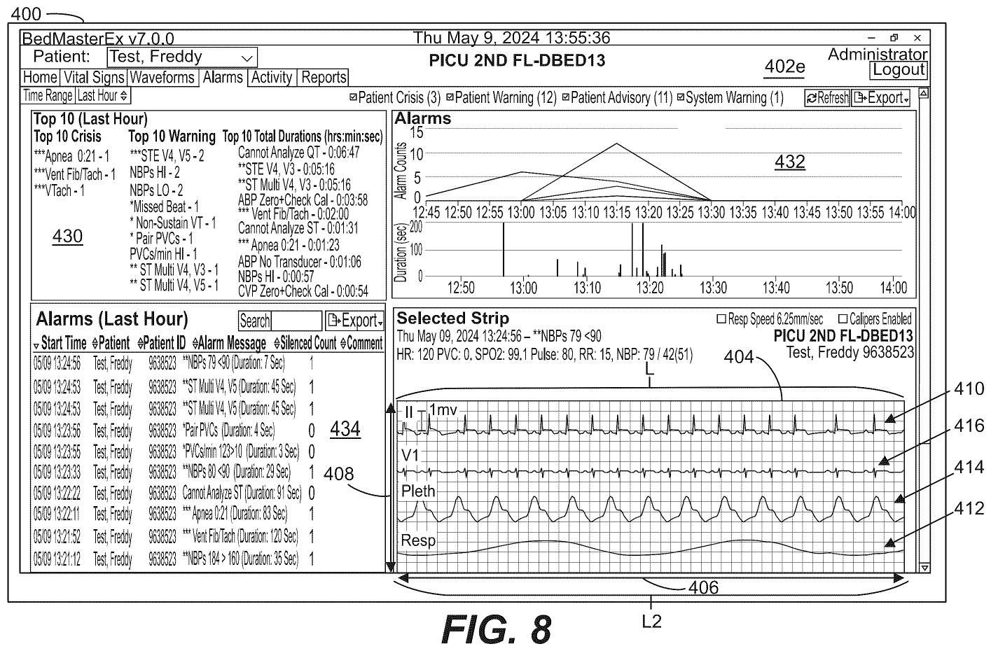

FIG. 8 illustrates another example of a graphical user interface 402e that can be displayed on the workstation monitor 400 by the data visualization system 200. The graphical user interface 402e is another example of a report page that can be generated by the data visualization system 200. In this example, the report page includes additional information based on the selections in the settings menu 420 (see FIGS. 5 and 6). For example, in addition to the waveform display area 404, the graphical user interface 402e further includes an alarm summary portion 430, an alarm charting portion 432, and an alarm composite table 434.

The alarm summary portion 430 summarizes alarms over a period of time (e.g., 1 hour). For example, the alarm summary portion 430 summarizes the most frequently issued alarms issued over the last hour for the patient P. Also, the alarm summary portion 430 can list the longest lasting alarms based on duration before being answered, suppressed, and/or delayed.

The alarm charting portion 432 graphically displays one or more charts such as a first chart showing an alarm counts (Y-axis) over time (X-axis). The alarm charting portion 432 can also display a second chart such as alarm duration (Y-axis) over time (X-axis).

The alarm composite table 434 can list all alarms issued over a period of time (e.g., 1 hour). For example, the alarms can be listed in chronological order and can include data such as start time, end time, patient name, patient ID, alarm message, silenced count, and comments which can be filtered by an end user as desired to display a subset of the alarms.

In the example shown in FIG. 8, the waveform display area 404 includes the first waveform 410, the second waveform 412, the third waveform 414, and the fourth waveform 416. As discussed above, the playback speed of the second waveform 412 can be adjusted independently of the playback of the first, third, and fourth waveforms 410, 414, 416 based on the selections in the settings menu 420 (see FIGS. 5 and 6). In this example, the playback speed of the second waveform 412 is adjusted to match the playback speeds of the first, third, and fourth waveforms 410, 414, 416 (i.e., the settings adjustment 422 is unchecked, see FIG. 5) such that the length L2 of the second waveform 412 matches the lengths L of the first, third, and fourth waveforms 410, 414, and 416, which causes the second waveform 412 to have the stretched appearance over the X-axis 406 in the waveform display area 404.

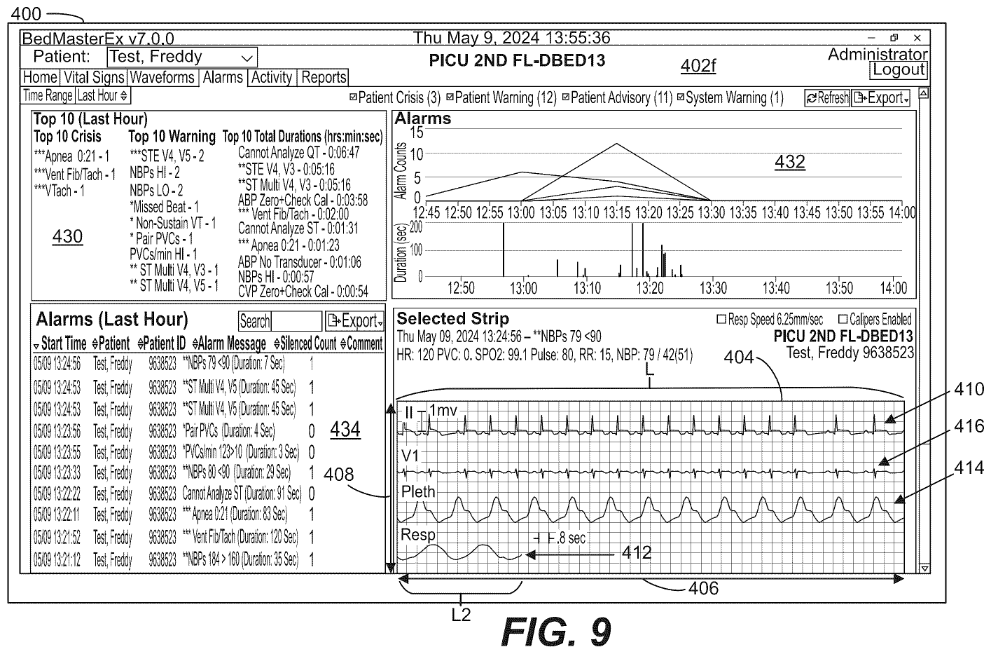

FIG. 9 illustrates another example of a graphical user interface 402f that can be displayed on the workstation monitor 400 by the data visualization system 200. The graphical user interface 402f is another example of a report page that can be generated by the data visualization system 200 that is similar to the graphical user interface 402e of FIG. 8.

The graphical user interface 402f differs from the graphical user interface 402e in that the length L2 of the second waveform 412 is shorter than the lengths L of the first, third, and fourth waveforms 410, 414, and 416, which can be based on the selections in the settings menu 420 such as when the settings adjustment 422 is checked (see FIG. 6). As discussed above, the playback speed of the second waveform 412 can be adjusted independently of the playback speeds of the first, third, and fourth waveforms 410, 414, 416. In this example, the playback speed of the second waveform 412 (e.g., 6.25 mm/second) is slower than the playback speeds of the first, third, and fourth waveforms 410, 414, and 416 (e.g., 25 mm/second) such that the length L2 of the second waveform 412 is shorter over a given period of time (e.g., 10 seconds) displayed on the X-axis 406 in the waveform display area 404.

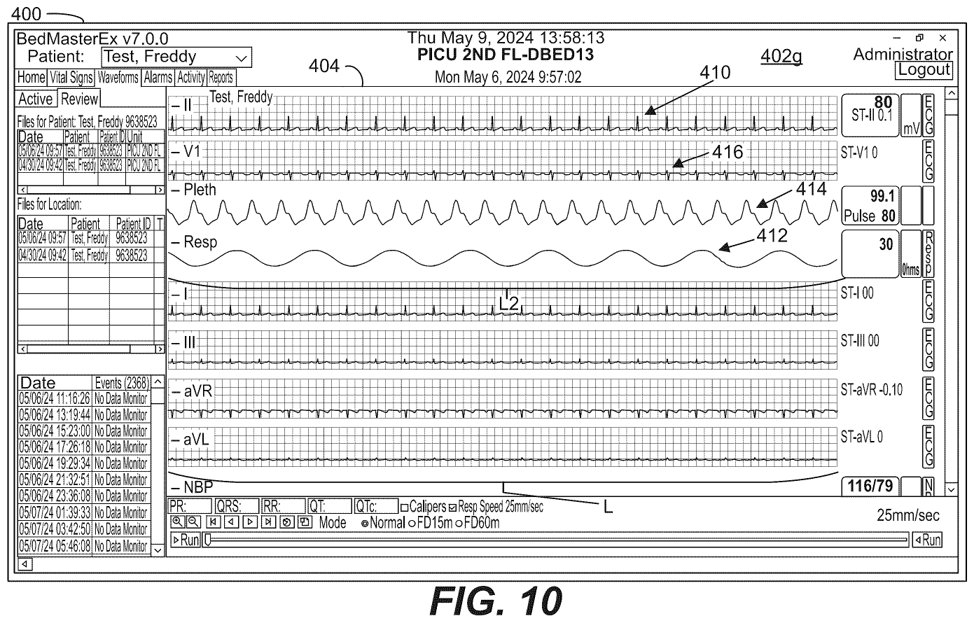

FIG. 10 illustrates another example of a graphical user interface 402g that can be displayed on the workstation monitor 400 by the data visualization system 200. The graphical user interface 402f is an example of a waveforms review tab that can be generated by the data visualization system 200. The waveforms review tab allows viewing the plurality of waveforms retrospectively on a page by page basis. The display of the waveforms review tab is designed to mimic the display 106 on the patient monitoring device 104 but also provide similar playback capabilities to that of a television DVR and easy navigation to events depicted in the waveforms.

As shown in FIG. 10, the length L2 of the second waveform 412 is equal to the lengths L of the first, third, and fourth waveforms 410, 414, and 416. Thus, the second waveform 412 has a stretched appearance that is visually different from the respiration rate waveforms displayed on the display 106 of the patient monitoring device 104. However, by have the length L2 of the second waveform equal to the lengths L of the first, third, and fourth waveforms 410, 414, 416, comparisons between the plurality of waveforms is made easier.

In the example shown in FIG. 10, the waveforms review tab displays additional types of waveforms in addition to the first, second, third, and fourth waveforms 410, 412, 414, and 416. For example, a fifth waveform is displayed in the waveform display area 404 representing a fifth physiological parameter such as an electrocardiogram signal captured by a lead III of the EKG sensor module 120 of the patient monitoring device 104; a sixth waveform is displayed in the waveform display area 404 representing a sixth physiological parameter such as an electrocardiogram signal captured by a lead aVR of the EKG sensor module 120 of the patient monitoring device 104; a seventh waveform is displayed in the waveform display area 404 representing a seventh physiological parameter such as an electrocardiogram signal captured by a lead aVL of the EKG sensor module 120 of the patient monitoring device 104; and an eighth waveform is displayed in the waveform display area 404 representing a eighth physiological parameter such as a non-invasive blood pressure captured by the NIBP sensor module 124 of the patient monitoring device 104. Additional types of waveforms, or fewer types of waveforms, can be displayed in the in the waveform display area 404 of the waveforms review tab as desired.

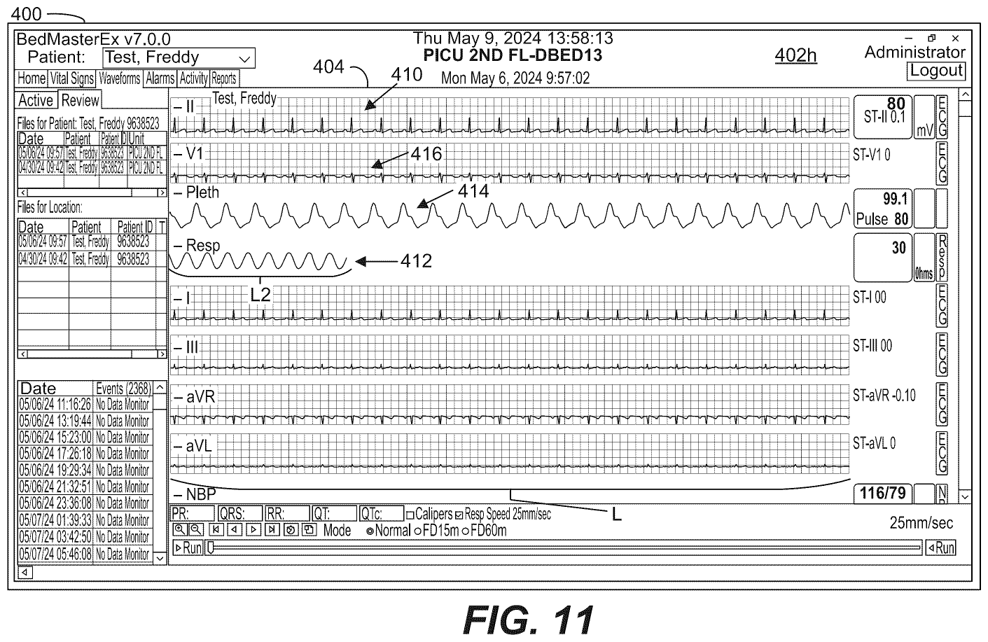

FIG. 11 illustrates another example of a graphical user interface 402h that can be displayed on the workstation monitor 400 by the data visualization system 200. The graphical user interface 402h is another example of a waveforms review tab that can be generated by the data visualization system 200. The graphical user interface 402h is similar to the graphical user interface 402g of FIG. 10 except the length L2 of the second waveform 412 is shorter than the lengths L of the other waveforms displayed in the waveform display area 404. As described above, the playback speed of the second waveform 412 can be adjusted independently of the playback speed of the other waveforms in the waveform display area 404. In this example, the playback speed of the second waveform 412 is slower than the playback speed of the other waveforms in the waveform display area 404 (i.e., by checking the settings adjustment 422, see FIG. 6) such that the length L2 of the second waveform 412 is shorter than the lengths L of the other waveforms over a given period of time along the X-axis in the waveform display area 404. In this example, the second waveform 412 has a visual appearance that is similar to the respiration rate waveforms displayed on the display 106 of the patient monitoring device 104.

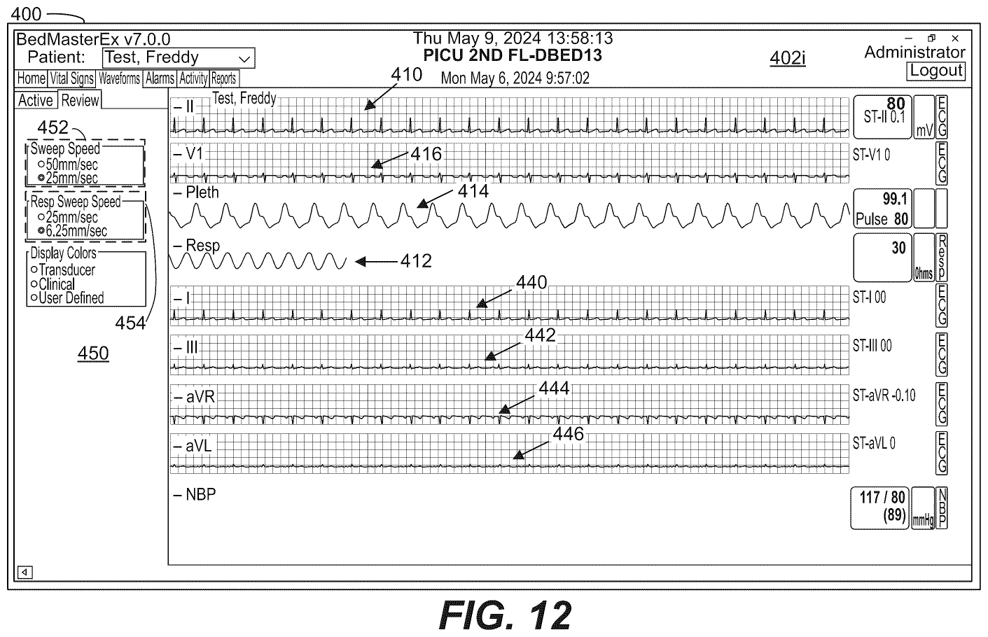

FIG. 12 illustrates another example of a graphical user interface 402i that can be displayed on the workstation monitor 400 by the data visualization system 200. The graphical user interface 402i is an example of an active waveforms page that displays in near real-time a plurality of waveforms captured by medical devices in the patient environment 100 such as the patient support apparatus 102, the patient monitoring device 104, and other medical devices.

The plurality of waveforms are displayed in near real-time in the waveform display area 404 which includes the first, second, third, and fourth waveforms 410, 412, 414, and 416 shown in the example graphical user interfaces of FIGS. 4-11. Additionally, the waveform display area 404 displays in near real-time a fifth waveform 440 of an electrocardiogram signal captured by the lead I of the EKG sensor module 120; a sixth waveform 442 of an electrocardiogram signal captured by the lead III of the EKG sensor module 120; a seventh waveform 444 of an electrocardiogram signal captured by the lead aVR of the EKG sensor module 120; and an eighth waveform 446 of an electrocardiogram signal captured by the lead aVL of the EKG sensor module 120. Additional types of waveforms, or fewer types of waveforms, can be displayed in the in the waveform display area 404 as desired.

The graphical user interface 402i includes a settings tab 450 that includes a first sweep speed settings adjustment 452 that is adjustable between two or more sweep speeds. For example, the first sweep speed settings adjustment 452 can be toggled between a sweep speed of 50 mm/second and a sweep speed of 25 mm/second. A selection of the sweep speed of 50 mm/second or the sweep speed of 25 mm/second in the first sweep speed settings adjustment 452 adjusts the time to complete one sweep of data for a sensor module in the patient environment 100 other than the respiration sensor module 130. For example, toggling between the two or more sweep speeds in the first sweep speed settings adjustment 452 causes the speed at which the EKG sensor module 120, the CVP sensor module 122, the NIBP sensor module 124, the SpO2 sensor module 126, and/or the temperature sensor module 128 of the patient monitoring device 104 record the waveform data for display in the waveform display area 404. Also, toggling between the two or more sweep speeds in the first sweep speed settings adjustment 452 can cause the speed at which the heart rate sensor module 132 of the patient support apparatus 102 records the waveform data for display in the waveform display area 404. By adjusting the sweep speed in the first sweep speed settings adjustment 452, the near real-time display of the waveforms 410, 414, 416, and 440-446 in the waveform display area 404 is adjusted such that the tracing of these waveforms increases or decreases in speed over time along the X-axis.

The settings tab 450 further includes a second sweep speed settings adjustment 454 that can be adjusted between two or more sweep speeds. For example, the second sweep speed settings adjustment 454 can be toggled between a sweep speed of 25 mm/second or a sweep speed of 6.25 mm/second. Selecting either the sweep speed of 25 mm/second or the sweep speed of 6.25 mm/second adjusts the time to complete one sweep of data for the respiration sensor module 130, and thus affects the playback of the second waveform 412. By adjusting the sweep speed in the second sweep speed settings adjustment 454, the near real-time display of the second waveform 412 in the waveform display area 404 is adjusted such that the tracing of the second waveform 412 increases or decreases in speed over time along the X-axis.

In the illustrative example shown in FIG. 12, the first sweep speed settings adjustment 452 is toggled to a sweep speed of 25 mm/second and the second sweep speed settings adjustment 454 is toggled to a sweep speed of 6.25 mm/second. In this example, the second waveform 412 is slower than the other waveforms displayed inside the waveform display area 404. Accordingly, the second waveform 412 has a shorter length for a given period of time (e.g., 10 seconds) than the other waveforms displayed inside the waveform display area 404.

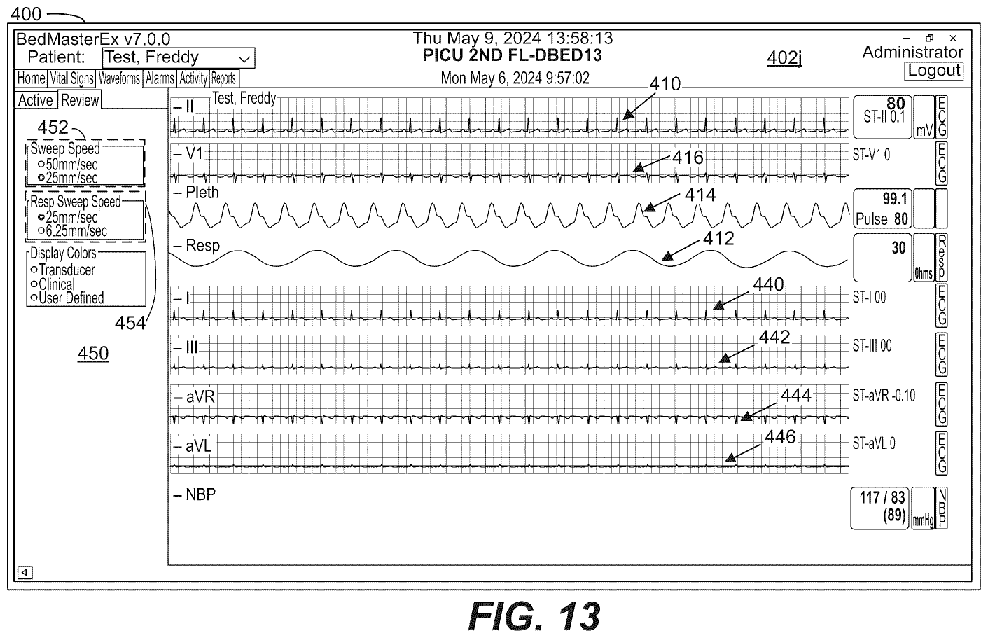

FIG. 13 illustrates another example of a graphical user interface 402j that can be displayed on the workstation monitor 400 by the data visualization system 200. The graphical user interface 402j is substantially similar to the graphical user interface 402i of FIG. 12, except in the example shown in FIG. 13, the first sweep speed settings adjustment 452 is toggled to a sweep speed of 25 mm/second and the second sweep speed settings adjustment 454 is toggled to a sweep speed of 25 mm/second. In FIG. 13, the second waveform 412 has the same sweep speed as the other waveforms displayed inside the waveform display area 404. Accordingly, the second waveform 412 has the same length for a given period of time (e.g., 10 seconds) as the other waveforms displayed inside the waveform display area 404.

The various embodiments described above are provided by way of illustration only and should not be construed to be limiting in any way. Various modifications can be made to the embodiments described above without departing from the true spirit and scope of the disclosure.

What is claimed is:

1. A device for displaying physiological parameter measurements, the device comprising:
at least one processing device; and
at least one computer-readable data storage device storing software instructions that, when executed by the at least one processing device, cause the at least one processing device to:
display a graphical user interface including playback of a plurality of waveforms, each waveform of the plurality of waveforms representing a physiological parameter measured over a period of time, the plurality of waveforms including at least:
a first waveform representing a first physiological parameter; and
a second waveform representing a second physiological parameter;
receive a settings adjustment on the graphical user interface; and
adjust the playback of the second waveform relative to the first waveform based on the settings adjustment, wherein the playback of the second waveform is adjusted to change a sweep speed of the second waveform such that a length of the second waveform differs from a length of the first waveform over the period of time.

2. The device of claim 1, wherein the first physiological parameter is an electrocardiogram signal, and the second physiological parameter is respiration rate.

3. The device of claim 2, wherein the graphical user interface further includes a third waveform representing a third physiological parameter; and
wherein the instructions, when executed by the at least one processing device, further cause the at least one processing device to:
adjust the playback of the second waveform relative to the third waveform based on the settings adjustment such that the length of the second waveform differs from a length of the third waveform over the period of time.

4. The device of claim 3, wherein the third waveform is pulsatile arterial blood flow.

5. The device of claim 3, wherein the graphical user interface further includes a fourth waveform representing a fourth physiological parameter; and
wherein the instructions, when executed by the at least one processing device, further cause the at least one processing device to:
adjust the playback of the second waveform relative to the fourth waveform based on the settings adjustment such that the length of the second waveform differs from a length of the fourth waveform over the period of time.

6. The device of claim 5, wherein the fourth waveform is a second electrocardiogram signal.

7. The device of claim 6, wherein the instructions, when executed by the at least one processing device, further cause the at least one processing device to:
receive a second settings adjustment on the graphical user interface; and
adjust the playback of the second waveform relative to the first, third, and fourth waveforms based on the second settings adjustment, the playback of the second waveform being adjusted to increase the sweep speed of the second waveform such that the length of the second waveform matches the lengths of the first, third, and fourth waveforms over the period of time.

8. The device of claim 1, wherein the sweep speed of the second waveform is decreased from 25 mm/second to 6.25 mm/second.

9. A method of displaying physiological parameter measurements, the method comprising:
displaying a graphical user interface including playback of a plurality of waveforms, each waveform of the plurality of waveforms representing a physiological parameter measured over a period of time, the plurality of waveforms including at least:
a first waveform representing a first physiological parameter; and
a second waveform representing a second physiological parameter;
receiving a settings adjustment on the graphical user interface; and
adjusting the playback of the second waveform relative to the first waveform based on the settings adjustment, the playback of the second waveform being adjusted to change a sweep speed of the second waveform such that a length of the second waveform differs from a length of the first waveform over the period of time.

10. The method of claim 9, wherein the first physiological parameter is an electrocardiogram signal, and the second physiological parameter is respiration rate.

11. The method of claim 10, further comprising:
displaying in the graphical user interface a third waveform representing a third physiological parameter; and
adjusting the playback of the second waveform relative to the third waveform based on the settings adjustment such that the length of the second waveform differs from a length of the third waveform over the period of time.

12. The method of claim 11, wherein the third waveform is pulsatile arterial blood flow.

13. The method of claim 11, further comprising:
displaying in the graphical user interface a fourth waveform representing a fourth physiological parameter; and
adjusting the playback of the second waveform relative to the fourth waveform based on the settings adjustment such that the length of the second waveform differs from a length of the fourth waveform over the period of time.

14. The method of claim 13, wherein the fourth waveform is a second electrocardiogram signal.

15. The method of claim 14, further comprising:
receiving a second settings adjustment on the graphical user interface; and
adjusting the playback of the second waveform relative to the first, third, and fourth waveforms based on the second settings adjustment, the playback of the second waveform being adjusted to increase the sweep speed of the second waveform such that the length of the second waveform matches the lengths of the first, third, and fourth waveforms over the period of time.

16. The method of claim 9, wherein the sweep speed of the second waveform is decreased from 25 mm/second to 6.25 mm/second.

17. Non-transitory computer-readable media storing data instructions, which when executed by one or more processing devices, cause the one or more processing devices to:
display a graphical user interface including playback of a plurality of waveforms, each waveform of the plurality of waveforms representing a physiological parameter measured over a period of time, the plurality of waveforms including at least:
a first waveform representing a first physiological parameter; and
a second waveform representing a second physiological parameter;
receive a settings adjustment on the graphical user interface; and
adjust the playback of the second waveform relative to the first waveform, wherein the playback of the second waveform is adjusted to change a sweep speed of the second waveform such that a length of the second waveform differs from a length of the first waveform over the period of time.

18. The non-transitory computer-readable media of claim 17, further comprising additional data instructions, which when executed by the one or more processing devices, further cause the one or more processing devices to:
display in the graphical user interface a third waveform representing a third physiological parameter and a fourth waveform representing a fourth physiological parameter; and
adjust the playback of the second waveform relative to the third and fourth waveforms such that the length of the second waveform differs from lengths of the third and fourth waveforms over the period of time.

19. The non-transitory computer-readable media of claim 18, wherein the first physiological parameter is a first electrocardiogram signal, the second physiological parameter is respiration rate, the third waveform is pulsatile arterial blood flow, and the fourth waveform is a second electrocardiogram signal.

20. The non-transitory computer-readable media of claim 18, further comprising additional data instructions, which when executed by the one or more processing devices, further cause the one or more processing devices to:
receive a second settings adjustment on the graphical user interface; and
adjust the playback of the second waveform relative to the first, third, and fourth waveforms based on the second settings adjustment, the playback of the second waveform being adjusted to increase the sweep speed of the second waveform such that the length of the second waveform matches the lengths of the first, third, and fourth waveforms over the period of time.
